(19) United States
(12) Patent Application Publication
(10) Pub. No.: US 2025/0090365 A1
(43) Pub. Date: Mar. 20, 2025

(54) Shield Device

(71) Applicant: Latoyia Armstead, St. Paul, MN (US)

(72) Inventor: Latoyia Armstead, St. Paul, MN (US)

(21) Appl. No.: 18/827,965

(22) Filed: Sep. 9, 2024

Related U.S. Application Data
(60) Provisional application No. 63/583,651, filed on Sep. 19, 2023.

(51) Int. Cl. A61F 5/443; A61F 5/44

(52) U.S. Cl. CPC A61F 5/443; A61F 5/4404

(57) ABSTRACT

A shield device is disclosed, which provides a user with a more discrete method of wearing medical products. The shield device comprises a body component that is configured in the shape of the ostomy bag. The body component is opaque with an adhesive on the rear side, which secures it to the ostomy bag. The body component is attached to the outside of a medical supply pouch, or it may be worn as a cover up to shield medical supplies from the view of others. The body component can be customized with different colors and designs. The device will essentially allow medical personnel to carry their supplies in a more discrete fashion.

### CROSS-REFERENCE TO RELATED APPLICATION

The present application claims priority to, and the benefit of, U.S. Provisional Application No. 63/583,651, which was filed on Sep. 19, 2023, and is incorporated herein by reference in its entirety.

### FIELD OF THE INVENTION

The present invention relates generally to the field of shield devices. More specifically, the present invention relates to a shield or sticker that covers the face of an ostomy bag and helps keep the equipment hidden and discreet. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in shield devices. Generally, people who wear an ostomy bag or other medical products may lack the means to keep the equipment hidden and discreet. Internal contents of the bag may be seen which can be embarrassing. Ostomy bag wearers may not be confident in their appearance if there is a risk of the bag being seen.

A surgical procedure known as a colostomy is performed on people in whom some portion of their large intestine has become inoperative due to disease or injury. A passageway, or stoma, is surgically formed on the patient through the abdominal wall, and the intestine is re-routed to the stoma. Waste material exits the body through this stoma, and a collection bag or appliance is attached to receive the waste material. There are a variety of collection bags in common use, but these all typically are made of a transparent or semitransparent plastic material and have a flange or fitting at the upper part of the bag to fasten removably to the stoma. In most cases, at the bottom of the collection bag there is a drain tube that can be opened to release fluid wastes.

There is some discomfort involved in the wearing of a colostomy bag. The bag itself is made of a plastic material, and this can irritate the patient's skin if in direct contact. Also, the bag can be somewhat cold to the touch when attached and warm to hot as it fills, and thus can be rather uncomfortable. The plastic material can also stick to the skin. Further, the bag is transparent and thus the waste material within can be seen, which can be embarrassing for the user.

Accordingly, there is a demand for an improved shield device that covers the face of an ostomy bag to keep the equipment hidden and discreet. More particularly, there is a demand for a shield device that offers a way for people who carry around medical equipment like an ostomy bag to have more confidence in their appearance.

Therefore, there exists a long felt need in the art for a shield device that provides users with a shield or sticker that covers the face of an ostomy bag. There is also a long felt need in the art for a shield device that helps keep the equipment hidden and discreet. Further, there is a long felt need in the art for a shield device that functions as a cover for several other different types of medical products to carry supplies in a more confident fashion. Moreover, there is a long felt need in the art for a device that enables users to secure the shield to the bag or other equipment via a durable adhesive. Further, there is a long felt need in the art for a shield device that offers a way for people who carry around medical equipment like an ostomy bag to have more confidence in their appearance. Finally, there is a long felt need in the art for a shield device that can be customized with different colors and designs.

The subject matter disclosed and claimed herein, in one embodiment thereof, comprises a shield device. The device provides a user with a more discrete method of wearing medical products. The shield device comprises a body component that is configured in the shape of the ostomy bag. The body component is opaque with an adhesive on the rear side, which secures it to the ostomy bag. The body component is attached to the outside of a medical supply pouch, or it may be worn as a cover up to shield medical supplies from the view of others. The body component can be customized with different colors and designs. The device will essentially allow medical personnel to carry their supplies in a more discrete fashion.

In this manner, the shield device of the present invention accomplishes all of the forgoing objectives and provides users with a device that provides a more discrete means for wearing medical products. The device is a shield that can be applied with adhesive to a ostomy bag. The device comes in different colors and designs.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a shield device. The device provides a user with a more discrete method of wearing medical products. The shield device comprises a body component that is configured in the shape of the ostomy bag. The body component is opaque with an adhesive on the rear side, which secures it to the ostomy bag. The body component is attached to the outside of a medical supply pouch, or it may be worn as a cover up to shield medical supplies from the view of others. The body component can be customized with different colors and designs. The device will essentially allow medical personnel to carry their supplies in a more discrete fashion.

Typically, the shield device is utilized to hide and shield medical products or supplies, such as colostomy bags, collection pouches for urine, etc., or any other suitable medical product as is known in the art that a user wishes to have discreetly hidden. Generally, the term “ostomy” comprehends both colostomy and ileostomy (which involves the small intestine). In both conditions, collection pouches are used, and in both conditions there are similar problems of discomfort and embarrassment. Similar collection bags are also used for urine collection.

Generally, the shield device is utilized with an ostomy bag and will be described with such, however, the device is not limited to use with only an ostomy bag and can be utilized with any suitable medical product as is known in the art, depending on the needs and/or wants of a user. Typically, the ostomy bag is made of a clear or transparent plastic film and has a fitting or flange on its upper portion which is secured to a stoma on the patient's abdomen. A drain tube is provided at the bottom of the ostomy bag for withdrawing liquid or fluid wastes from the ostomy bag. The drain tube has a closure or stopper that can be removed to permit drainage of the ostomy bag.

In one embodiment, the shield device comprises a body component. The body component is made from a single rectangle of material cut to cover the entire front of an ostomy bag. The body component is then cut to mirror the shape and size of the ostomy bag. The body component can be sized approximately six to twelve inches in length and approximately three to six inches in width to sufficiently cover the entire front of an ostomy bag and block it from view. The body component can be available in different sizes, depending on the size of the ostomy bag it is to cover. For example, the ostomy bag can be mini, midi, or maxi in size. A mini ostomy bag is smaller and more discreet, often used for short periods or during activities like exercise. A midi ostomy bag is medium in size and offers a balance between discretion and capacity. A maxi bag is a larger bag and provides more capacity, often used overnight or for those with higher output. The body component can be available in different sizes to fit any of these ostomy bag sizes. Typically, the body component can be any suitable shape and size as is known in the art, as long as it is sized and shaped to cover the front side of an ostomy bag from view.

In one embodiment, the body component comprises a front surface and a rear surface. The front surface is opaque, such that when positioned in front of the ostomy bag, view of the ostomy bag is blocked. In one embodiment, the front surface of the body component comprises various colors, patterns and designs and other aesthetic characteristics depending on the needs and/or wants of a user.

In one embodiment, the rear surface of the body component comprises an adhesive, which allows the body component to be releasably secured to the ostomy bag and removed as needed. Any suitable securing means can be utilized as is known in the art. In use, the body component is attached to the outside of the ostomy bag to block its view.

In one embodiment, the body component is a fabric cover. The fabric cover is made entirely of a soft, absorbent cloth. This is the same material that is often used in undergarments, i.e., tee shirts, and has a high degree of user comfort. The material can also be easily laundered and kept clean and sanitary. In this case, the ostomy bag and cover are worn outside an undergarment, but optionally the cover could be incorporated into the user's undergarment.

In one embodiment, the fabric cover is made from a single rectangle of material cut about eleven inches by about thirteen inches. The cover is formed of an outer or distal panel that covers the side of the ostomy bag away from the user, and left and right proximal panels, that meet at a vertical midline to cover the proximal or user side of the ostomy bag. The two proximal panels are joined by a stitched seam below a point partway down from a top seam of the cover with a slot. This slot provides access for installing the ostomy bag inside the cover, and also permits the fitting or flange to protrude. Another slot provides access to the drain tube, so that accumulated waste can be withdrawn from the ostomy bag without removing the cover. The fabric cover is positioned between the patient and the ostomy bag to prevent contact and help avoid skin irritation from contact with the plastic film, and discomfort due to heat or cold.

In yet another embodiment, the shield device comprises a plurality of indicia.

In yet another embodiment, a method of covering an ostomy bag is disclosed. The method includes the steps of providing a shield device comprising a body component with an adhesive back. The method also comprises choosing a body component of a specific color and design. Further, the method comprises removing a release liner from the adhesive back. The method also comprises securing the body component to an ostomy bag via the adhesive back. Finally, the method comprises covering the ostomy bag with the device in a discrete fashion.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the shield device of the present invention showing the opaque side in accordance with the disclosed architecture;

FIG. 2 illustrates a perspective view of one embodiment of the shield device of the present invention showing the adhesive side in accordance with the disclosed architecture;

FIG. 3 illustrates a perspective view of one embodiment of the shield device of the present invention showing the device in different colors and designs in accordance with the disclosed architecture;

FIG. 4 illustrates a perspective view of one embodiment of the shield device of the present invention showing the device in use in accordance with the disclosed architecture; and

FIG. 5 illustrates a flowchart showing the method of covering an ostomy bag in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a shield device that provides users with a shield or sticker that covers the face of an ostomy bag. There is also a long felt need in the art for a shield device that helps keep the equipment hidden and discreet. Further, there is a long felt need in the art for a shield device that functions as a cover for several other different types of medical products to carry supplies in a more confident fashion. Moreover, there is a long felt need in the art for a device that enables users to secure the shield to the bag or other equipment via a durable adhesive. Further, there is a long felt need in the art for a shield device that offers a way for people who carry around medical equipment like an ostomy bag to have more confidence in their appearance. Finally, there is a long felt need in the art for a shield device that can be customized with different colors and designs.

The present invention, in one exemplary embodiment, is a novel shield device. The shield device comprises a body component that is configured in the shape of the ostomy bag. The body component is opaque with an adhesive on the rear side, which secures it to the ostomy bag. The body component is attached to the outside of a medical supply pouch, or it may be worn as a cover up to shield medical supplies from the view of others. The body component can be customized with different colors and designs. The present invention also includes a novel method of covering an ostomy bag. The method includes the steps of providing a shield device comprising a body component with an adhesive back. The method also comprises choosing a body component of a specific color and design. Further, the method comprises removing a release liner from the adhesive back. The method also comprises securing the body component to an ostomy bag via the adhesive back. Finally, the method comprises covering the ostomy bag with the device in a discrete fashion.

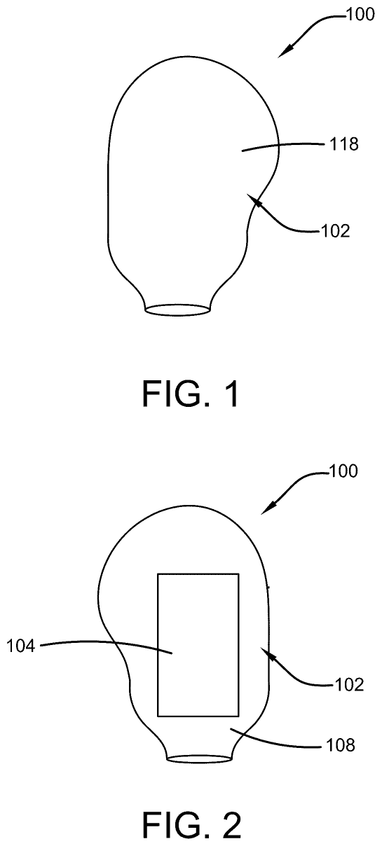

Referring initially to the drawings, FIG. 1 illustrates a perspective view of one embodiment of the shield device 100 of the present invention. In the present embodiment, the shield device 100 is an improved shield device 100 that provides a user 106 with a covering for an ostomy bag 110. Specifically, the shield device 100 comprises a body component 102. The body component 102 is opaque with an adhesive 104 on the rear side 108, which secures it to the ostomy bag 110. The body component 102 is attached to the outside 112 of a medical supply pouch 110, or it may be worn as a cover up to shield medical supplies from the view of others. The device 100 will essentially allow medical personnel to carry their supplies in a more discrete fashion.

Typically, the shield device 100 is utilized to hide and shield medical products or supplies, such as colostomy bags 110, collection pouches for urine, etc., or any other suitable medical product as is known in the art that a user 106 wishes to have discreetly hidden. Generally, the term “ostomy” comprehends both colostomy and ileostomy (which involves the small intestine). In both conditions, collection pouches 110 are used, and in both conditions there are similar problems of discomfort and embarrassment. Similar collection bags are also used for urine collection.

Generally, the shield device 100 is utilized with an ostomy bag 110 and will be described with such, however, the device 100 is not limited to use with only an ostomy bag 110 and can be utilized with any suitable medical product as is known in the art, depending on the needs and/or wants of a user 106. Typically, the ostomy bag 110 is made of a clear or transparent plastic film and has a fitting or flange 114 on its upper portion which is secured to a stoma on the patient's abdomen. A drain tube 116 is provided at the bottom of the ostomy bag 110 for withdrawing liquid or fluid wastes from the ostomy bag 110. The drain tube 116 has a closure or stopper that can be removed to permit drainage of the ostomy bag 110.

Further, the shield device 100 comprises a body component 102. The body component 102 is made from a single rectangle of material cut to cover the entire front of an ostomy bag 110. The body component 102 is then cut to mirror the shape and size of the ostomy bag 110. The body component 102 can be sized approximately six to twelve inches in length and approximately three to six inches in width to sufficiently cover the entire front of an ostomy bag 110 and block it from view. The body component 102 can be available in different sizes, depending on the size of the ostomy bag 110 it is to cover. For example, the ostomy bag 110 can be mini, midi, or maxi in size. A mini ostomy bag 110 is smaller and more discreet, often used for short periods or during activities like exercise. A midi ostomy bag 110 is medium in size and offers a balance between discretion and capacity. A maxi ostomy bag 110 is a larger bag and provides more capacity, often used overnight or for those with higher output. The body component 102 can be available in different sizes to fit any of these ostomy bag 110 sizes. Typically, the body component 102 can be any suitable shape and size as is known in the art, as long as it is sized and shaped to cover the front side of an ostomy bag 110 from view.

As shown in FIG. 2, the rear surface 108 of the body component 102 comprises an adhesive 104, which allows the body component 102 to be releasably secured to the ostomy bag 110 and removed as needed. Any suitable securing means can be utilized as is known in the art. In use, the body component 102 is attached to the outside of the ostomy bag 110 to block its view.

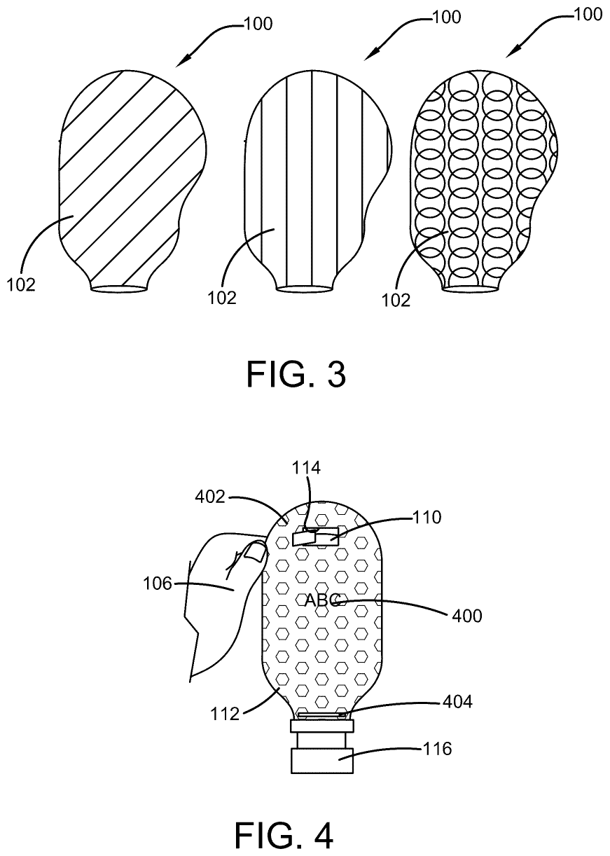

As shown in FIG. 3, the body component 102 comprises a front surface 118 and a rear surface 108. The front surface 118 is opaque, such that when positioned in front of the ostomy bag 110, view of the ostomy bag 110 is blocked. In one embodiment, the front surface 118 of the body component 102 comprises various colors, patterns and designs and other aesthetic characteristics depending on the needs and/or wants of a user 106.

As shown in FIG. 4, the body component 102 is a fabric cover 402. The fabric cover 402 is made entirely of a soft, absorbent cloth. This is the same material that is often used in undergarments, i.e., tee shirts, and has a high degree of user comfort. The material can also be easily laundered and kept clean and sanitary. In this case, the ostomy bag 110 and cover 402 are worn outside an undergarment, but optionally the cover 402 could be incorporated into the user's undergarment.

Further, the fabric cover 402 is made from a single rectangle of material cut about eleven inches by about thirteen inches. The cover 402 is formed of an outer or distal panel that covers the side of the ostomy bag 110 away from the user 106, and left and right proximal panels, that meet at a vertical midline to cover the proximal or user side of the ostomy bag 110. The two proximal panels are joined by a stitched seam below a point partway down from a top seam of the cover 402 with a slot 404. This slot 404 provides access for installing the ostomy bag 110 inside the cover 402, and also permits the fitting or flange 114 to protrude. Another slot 404 provides access to the drain tube 116, so that accumulated waste can be withdrawn from the ostomy bag 110 without removing the cover 402. The fabric cover 402 is positioned between the user 106 and the ostomy bag 110 to prevent contact and help avoid skin irritation from contact with the plastic film, and discomfort due to heat or cold.

In yet another embodiment, the shield device 100 comprises a plurality of indicia 400. The body component 102 of the device 100 may include advertising, a trademark, or other letters, designs, or characters, printed, painted, stamped, or integrated into the body component 102, or any other indicia 400 as is known in the art. Specifically, any suitable indicia 400 as is known in the art can be included, such as but not limited to, patterns, logos, emblems, images, symbols, designs, letters, words, characters, animals, advertisements, brands, etc., that may or may not be ostomy bag, shield, or brand related.

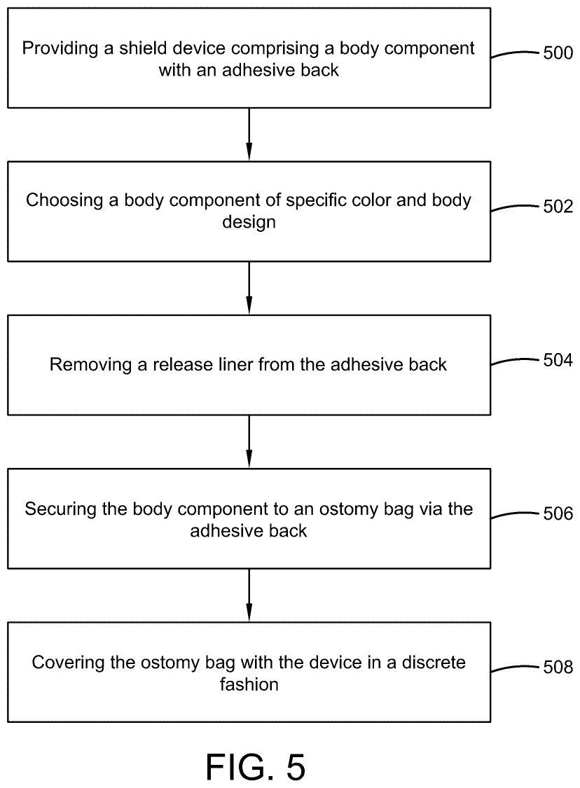

FIG. 5 illustrates a flowchart of the method of covering an ostomy bag. The method includes the steps of at 500, providing a shield device comprising a body component with an adhesive back. The method also comprises at 502, choosing a body component of a specific color and design. Further, the method comprises at 504, removing a release liner from the adhesive back. The method also comprises at 506, securing the body component to an ostomy bag via the adhesive back. Finally, the method comprises at 508, covering the ostomy bag with the device in a discrete fashion.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “shield device” and “device” are interchangeable and refer to the shield device 100 of the present invention.

Notwithstanding the forgoing, the shield device 100 of the present invention can be of any suitable size and configuration as is known in the art without affecting the overall concept of the invention, provided that it accomplishes the above stated objectives. One of ordinary skill in the art will appreciate that the shield device 100 as shown in FIGS. 1-5 is for illustrative purposes only, and that many other sizes and shapes of the shield device 100 are well within the scope of the present disclosure. Although the dimensions of the shield device 100 are important design parameters for user convenience, the shield device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A shield device that provides a user with a covering for an ostomy bag, the shield device comprising:
a body component, wherein the body component is opaque and secures to an ostomy bag to discreetly hide the ostomy bag from view during use.

2. The shield device of claim 1, wherein an ostomy bag comprises either colostomy and ileostomy bags.

3. The shield device of claim 2, wherein the ostomy bag is made of a clear or transparent plastic film and has a flange on its upper portion which is secured to a stoma on a user's abdomen and a drain tube at a bottom of the ostomy bag for withdrawing liquid or fluid wastes from the ostomy bag.

4. The shield device of claim 3, wherein the body component is made from a single rectangle of material cut to cover an entire front of the ostomy bag.

5. The shield device of claim 4, wherein the body component is cut to mirror shape and size of the ostomy bag.

6. The shield device of claim 5, wherein the body component is sized approximately six to twelve inches in length and approximately three to six inches in width to sufficiently cover the entire front of the ostomy bag and block it from view.

7. The shield device of claim 6, wherein the body component is available in different sizes, depending on size of the ostomy bag it is to cover.

8. The shield device of claim 7, wherein the body component has a front surface and a rear surface.

9. The shield device of claim 8, wherein the rear surface of the body component comprises an adhesive, which allows the body component to be releasably secured to the ostomy bag.

10. The shield device of claim 9, wherein the front surface of the body component is opaque, such that when positioned in front of the ostomy bag, view of the ostomy bag is blocked.

11. The shield device of claim 10, wherein the front surface of the body component comprises various colors, patterns and designs.

12. The shield device of claim 11 further comprising a plurality of indicia.

13. A shield device that provides a user with a covering for an ostomy bag, the shield device comprising:
a body component with a front surface and a rear surface;
wherein the body component is made from a single rectangle of material cut to cover an entire front of the ostomy bag;
wherein the body component is cut to mirror shape and size of the ostomy bag;
wherein the body component is sized approximately six to twelve inches in length and approximately three to six inches in width to sufficiently cover the ostomy bag;
wherein the rear surface of the body component comprises an adhesive, which allows the body component to be releasably secured to the ostomy bag; and
further wherein the front surface of the body component is opaque, such that when positioned in front of the ostomy bag, view of the ostomy bag is blocked.

14. The shield device of claim 13, wherein the body component is a fabric cover.

15. The shield device of claim 14, wherein the fabric cover encompasses an entire ostomy bag.

16. The shield device of claim 13 further comprising a plurality of indicia.

17. The shield device of claim 13, wherein the front surface of the body component comprises various colors, patterns and designs.

18. The shield device of claim 13, wherein the body component is available in different sizes, depending on size of the ostomy bag it is to cover.

19. The shield device of claim 13, wherein an ostomy bag comprises either colostomy and ileostomy bags.

20. A method of covering an ostomy bag, the method comprising the following steps:
providing a shield device comprising a body component with an adhesive back;
choosing a body component of a specific color and design;
removing a release liner from the adhesive back;
securing the body component to an ostomy bag via the adhesive back; and
covering the ostomy bag with the device in a discrete fashion.
